(19) United States
(12) Patent Application Publication
(10) Pub. No.: US 2023/0402186 A1
(43) Pub. Date: Dec. 14, 2023

(54) APPARATUS, METHOD AND COMPUTER READABLE STORAGE MEDIUM FOR PREDICTING BLOOD PRESSURE NON-COMPRESSIVELY USING CONVOLUTIONAL NEURAL NETWORK AND LONG-SHORT-TERM MEMORY NETWORK

(71) Applicant: Industry-Academic Cooperation Foundation ,Chosun University, Gwangiu (KR)

(72) Inventors: Youn Tae KIM, Daejeon (KR); Jae Hyo JUNG, Gwangju (KR); Si Ho SHIN, Gwangju (KR); Geng Jia ZHANG, Gwangju (KR)

(21) Appl. No.: 18/146,735

(22) Filed: Dec. 27, 2022

(30) Foreign Application Priority Data
Jun. 8, 2022 (KR) 10-2022-0069746

(51) Int. Cl. G16H 50/30; A61B 5/00; G06N 3/0442; G06N 3/0464

(52) U.S. Cl. CPC G16H 50/30; A61B 5/7264; A61B 5/7275; G06N 3/0442; G06N 3/0464

(57) ABSTRACT

An apparatus for predicting blood pressure non-compressively includes a sequence folding layer configured to convert a sequence image of non-pressurized biosignals into an arrayed image; a CNN layer configured to generate a feature map by performing a convolution operation on an arrayed image; a sequence unfolding layer configured to convert the generated feature map into a sequence image; a flatten layer configured to convert the converted sequence image into one-dimensional data; a long-short-term memory network layer configured to extract feature values from the converted one-dimensional data using weights; a fully-connected layer configured to perform image classification using feature values extracted from the long-short memory network layer; and a regression layer configured to predict systolic blood pressure (SBP) and diastolic blood pressure (DBP) for the classified image.

### CROSS-REFERENCE TO RELATED APPLICATION(S)

This application claims benefit of priority to Korean Patent Application No. 10-2022-0069746 filed on Jun. 8, 2022 in the Korean Intellectual Property Office, the disclosure of which is incorporated herein by reference in its entirety.

### BACKGROUND

### 1. Field

Embodiments of the present disclosure relate to an apparatus, a method and a computer readable storage medium for predicting blood pressure non-compressively using a convolutional neural network and a long-short-term memory network.

This application was derived from research undertaken as a part of a project [Project Number: 1711120116, Project Title: Development of Brain-Body Interface Technology Using AI-Based Multi-Sensing] to support the Institute for Information & Communications Technology and as a part of a project [Project Number: 1345350649, Project Title: Development of Key to wearable cuffless blood pressure measurements based on multiple biosignal] to support the Ministry of Education.

### 2. Description of Related Art

High blood pressure is a common and dangerous disease which has no warning signs or symptoms and is known as a “silent killer” because many people are unaware that they have high blood pressure. For this reason, it is important to check blood pressure regularly.

Blood pressure may be the most basic medical parameter indicating a health condition. As a method of measuring basic blood pressure, an oscillometric method using a cuff may be used. A blood pressure measuring system using an oscillometric method may be a device which may monitor blood pressure levels by measuring pressure exerted by a cuff on an artery of an arm.

A generally used oscillometric automatic blood pressure measuring system may use a method of calculating blood pressure by detecting an oscillation signal while first pressurizing up to a certain pressure quickly and then slowly reducing the pressure.

However, this method has disadvantages, in that excessive pressure may be applied to a person and the measurement time may be increased.

### SUMMARY

An embodiment of the present disclosure is to provide an apparatus, a method and a computer readable storage medium for predicting blood pressure non-compressively using a convolutional neural network and a long-short-term memory network

According to an embodiment of the present disclosure, an apparatus for predicting blood pressure non-compressively includes a sequence folding layer configured to convert a sequence image of non-pressurized biosignals into an arrayed image; a CNN layer configured to generate a feature map by performing a convolution operation on an arrayed image; a sequence unfolding layer configured to convert the generated feature map into a sequence image; a flatten layer configured to convert the converted sequence image into one-dimensional data; a long-short-term memory network layer configured to extract feature values from the converted one-dimensional data using weights; a fully-connected layer configured to perform image classification using feature values extracted from the long-short memory network layer; and a regression layer configured to predict systolic blood pressure (SBP) and diastolic blood pressure (DBP) for the classified image.

The non-pressurized biosignal may be a signal in which the number of pieces of data is increased using random cropping.

The non-pressurized biosignal may include an electrocardiogram (ECG) signal and a photoplethysmography (PPG) signal.

The CNN layer may include a first convolution layer configured to generate a first feature map by performing a convolution operation on the arrayed image; a first max pooling layer configured to reduce a dimension of the first feature map by extracting a maximum value of the generated first feature map; a second convolution layer configured to generate a second feature map by performing a convolution operation on the first feature map of which a dimension is reduced; and a second max pooling layer configured to reduce a dimension of the second feature map by extracting a maximum value of the generated second feature map.

The apparatus for predicting blood pressure non-compressively may further include a first normalization layer configured to normalize the first feature map between the first convolution layer and the first max pooling layer; and a second normalization layer configured to normalize the second feature map between the second convolution layer and the second max pooling layer.

According to an embodiment of the present disclosure, a method for predicting blood pressure non-compressively includes a first step of converting a sequence image of non-pressurized biosignals into an arrayed image in a sequence folding layer; a second step of generating a feature map by performing a convolution operation on an arrayed image in a CNN layer; a third step of converting the generated feature map into a sequence image in a sequence unfolding layer; a fourth step of converting the converted sequence image into one-dimensional data in a flatten layer; a fifth step of extracting feature values from the converted one-dimensional data using weights in a long-short-term memory network layer; a sixth step of performing image classification using feature values extracted from the long-short-term memory network layer in a fully connected layer; and a seventh step of predicting systolic blood pressure (SBP) and diastolic blood pressure (DBP) for the classified images in a regression layer.

According to an embodiment of the present disclosure, a computer readable storage medium in which a program for executing the method on a computer is written is provided.

### BRIEF DESCRIPTION OF DRAWINGS

The above and other aspects, features, and advantages of the present disclosure will be more clearly understood from the following detailed description, taken in conjunction with the accompanying drawings, in which:

FIG. 1 is a block diagram illustrating an apparatus for predicting blood pressure non-compressively according to an embodiment of the present disclosure;

FIGS. 2A and 2B are graphs illustrating an ECG signal and a PPG signal, which are non-pressurized biosignals, according to an embodiment of the present disclosure;

FIGS. 3A and 3B are graphs illustrating an error (RSME) between a predicted blood pressure and a measured blood pressure value according to an embodiment of the present disclosure;

FIGS. 4A and 4B are graphs illustrating performance of an apparatus for predicting blood pressure non-compressively using a Bland-Altman plot according to an embodiment of the present disclosure; and

FIG. 5 is a flowchart illustrating a method for predicting blood pressure non-compressively according to an embodiment of the present disclosure.

### DETAILED DESCRIPTION

Hereinafter, embodiments of the present disclosure will be described as below with reference to the attached drawings.

It is to be understood that various equivalents and modifications may replace the embodiments and configurations at the time of the present application. In the drawings, same elements will be indicated by same reference numerals. Also, redundant descriptions and detailed descriptions of known functions and elements that may unnecessarily make the gist of the present disclosure obscure will not be provided. In the accompanying drawings, some elements may be exaggerated, omitted or briefly illustrated, and the sizes of the elements do not necessarily reflect the actual sizes of these elements.

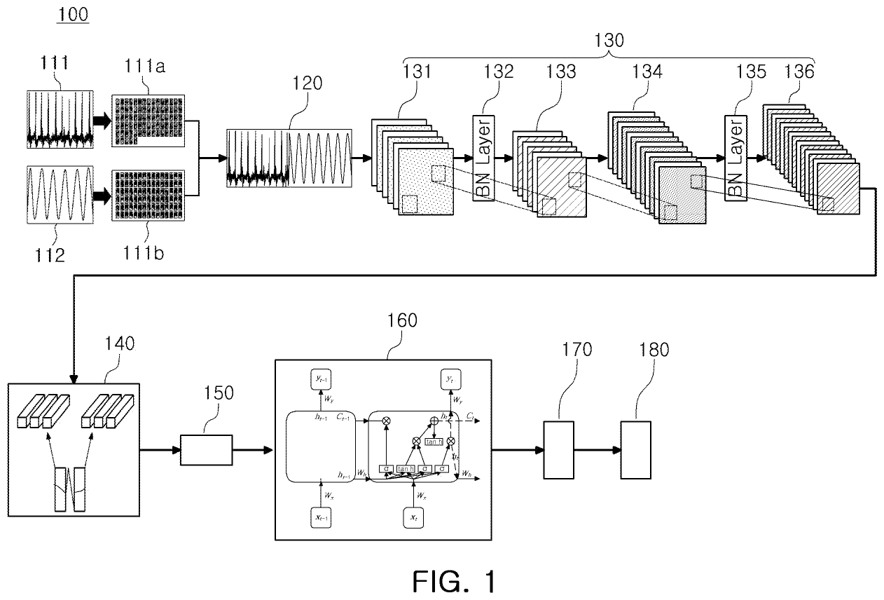

FIG. 1 is a block diagram illustrating an apparatus 100 for predicting blood pressure non-compressively according to an embodiment.

As illustrated in FIG. 1, the apparatus 100 for predicting non-pressurized blood pressure in an embodiment may include a sequence folding layer 120, a CNN layer 130, a sequence unfolding layer 140, a flatten layer 150, a long-short-term memory network layer 160, a fully connected layer 170, and a regression layer 180.

Specifically, the sequence folding layer 120 may convert a sequence image of non-pressurized bio-signals into an arrayed image. The converted array image may be transferred to the CNN layer 130. The aforementioned non-pressurized biosignal may include an electrocardiogram (ECG) signal 111 and a photoplethysmography (PPG) signal.

As illustrated in FIG. 1, the non-pressurized biosignals in the embodiment may be signals 111a and 112a in which the number of pieces of data is increased using random cropping. The aforementioned random cropping may be a method of increasing the number of pieces of data by randomly cropping or scaling image data. By increasing the number of pieces of data using random cropping as described above, overfitting may be prevented.

A convolutional neural network layer (CNN) layer 130 may generate a feature map by performing a convolution operation on the arrayed image.

Specifically, the CNN layer 130 may include a first convolution layer 131 generating a first feature map by performing a convolution operation on an arrayed image, a first max pooling layer 133 reducing a dimension of the first feature map by extracting a maximum value of the generated first feature map, a second convolution layer 134 generating a second feature map by performing a convolution operation on the dimensionally reduced first feature map, and a second max pooling layer 136 for reducing a dimension of the second feature map by extracting a maximum value of the generated second feature map.

Also, in an embodiment, a first normalization layer 132 for normalizing the first feature map may be further included between the first convolution layer 131 and the first max pooling layer 133, and a second normalization layer 135 for normalizing the second feature map may be further included between the second convolution layer 134 and the second max pooling layer 136.

The sequence unfolding layer 140 may convert the generated feature map into a sequence image. The converted sequence image may be transferred to the flatten layer 150.

The flatten layer 150 may convert the converted sequence image into one-dimensional data. The converted one-dimensional data may be transferred to the long-short-term memory network layer 160.

In an embodiment, using one-dimensional data output from the flatten layer 150 as an input value of the long-short-term memory network layer 160, it may not be necessary to convert parameters of the long-short-term memory network layer 160 described later.

The long-short-term memory network layer 160 may extract feature values from one-dimensional data using weights. The extracted feature values may be transferred to the fully connected layer 170.

That is, the long-short-term memory network layer 160 may extract a feature value according to Equation 1 below by applying a weight having a size of 800×16248 to the input layer.

iₜ=σ(wₓxₜ+wₕhₜ₋₁+b₁

gₜ=tanh(w_{xg}xₜ+w_{hg}hₜ₋₁+b_{g})

fₜ=σ(w_{xf}xₜ+w_{hf}hₜ₋₁+b_{f})

oₜ=σ(wₓₒxₜ+wₕₒhₜ₋₁+bₒ)

cₜ=fₜ·cₜ₋₁+iₜ·gₜ [Equation 1]

Equation 1 illustrates a process of operation of the long-short-term memory network layer 160. The long-short-term memory network layer 160 may include an input gate (iₜ, gₜ), a forget gate (fₜ), and an output gate (Oₜ). The input gate (iₜ, gₜ) may determine new information, the forget gate (fₜ) may determine previous information, and the output gate (Oₜ) may control an output value of the updated cell. In this case, each gate may multiply the weight value according to the input vector (xₜ), hidden state (hₜ₋₁), and cell state (Cₜ) using a sigmoid function and a hyperbolic tangent (tanh) function, and may calculate the feature value.

Thereafter, by applying equation 2 below, the feature value calculated at the output gate may be transferred to the output layer. That is, feature values from −1 to 1 may be extracted using the Tanh function, the feature values in the range calculated on the output gate may be transferred to the output layer.

hₜ=Oₜ·tanh(cₜ) [Equation 2]

The above-described long-short-term memory network layer 160 may be a type of recurrent neural networks (RNN), and may be an artificial neural network which may recognize a pattern in data having an array form such as text and gene signal analysis. In a general artificial neural network, when data is input, operations may be performed in sequence from an input layer through a hidden layer to an output. In this process, the input data may pass through the entirety of nodes only once. There may be a disadvantage of not remembering the previous data well.

However, differently from a general artificial neural network, RNN may be connected such that the result of the hidden layer may return to the input of the same hidden layer. Accordingly, the output of the hidden layer may be repeatedly input to the same hidden layer. However, large data may not be handled well, such that an operation speed may be lowered. To address the issue, a long short-term memory network (LSTM) may be used. A long-short-term memory network may be a special type of RNN, and may be a neural network designed to memorize and learn well even when a distance between sequential input data is long.

The fully connected layer 170 may connect feature values extracted from the long-short-term memory network layer 160 in a one-dimensional array form.

Finally, the regression layer 180 may predict systolic blood pressure (SBP) and diastolic blood pressure (DBP) through fitting of feature values connected in the form of a one-dimensional array. That is, the feature values connected in the form of a one-dimensional array in the fully connected layer 170 may generate optimal values through fitting in the regression data 180, and the generated values may be systolic blood pressure (SBP) and diastolic blood pressure (DBP).

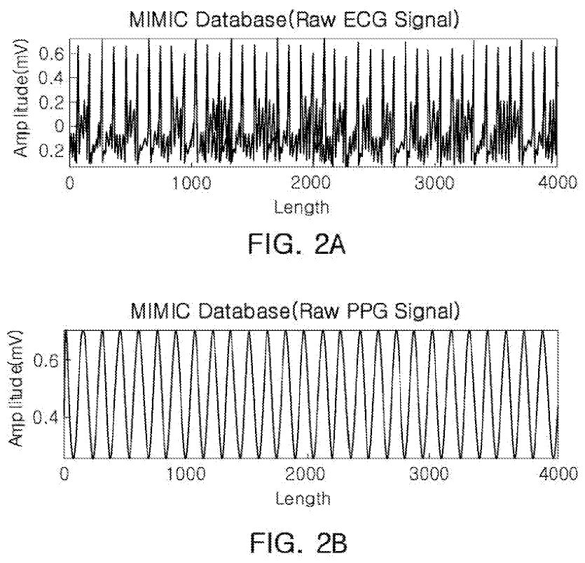

In the embodiment, the BIDMC Database and the MIMIC Database were used for learning. MIMIC Database and BIDMC Database may consist of electrocardiogram as shown in FIG. 2A and pulse wave signals as shown in FIG. 2B in a file, and 1,912 signals for 942 test subjects are recorded. The sampling frequency of each bio-signal may be 125 Hz.

The structure of each layer described above is summarized in table 1 below.

TABLE 1

| Number | Layer | Activations | Weights | Bias |
|---|---|---|---|---|
| 1 | Sequence Input | 150*150*3 | — | — |
| | Layer | | | |
| 2 | Sequence | 150*150*1 | — | — |
| | Folding Layer | | | |
| 3 | Convolution 2D | 150*150*6 | 5*5*3*6 | 1*1*6 |
| | Layer | | | |
| 4 | Batch | 150*150*6 | — | — |
| | Normalization | | | |
| | Layer | | | |
| 5 | Max Pooling | 75*75*6 | — | — |
| | Layer | | | |
| 6 | Convolution 2D | 75*75*12 | 3*3*6*12 | 1*1*12 |
| | Layer | | | |
| 7 | Batch | 75*75*12 | — | — |
| | Normalization | | | |
| | Layer | | | |
| 8 | Max Pooling | 37*37*12 | — | — |
| | Layer | | | |
| 9 | Sequence | 37*37*12 | — | — |
| | Unfolding Layer | | | |
| 10 | Flatten Layer | 16428 | — | — |
| 11 | LSTM | 200 | Input: | 800*1 |
| | | | 800*16428 | |
| | | | Recurrent: | |
| | | | 800*200 | |
| 12 | Fully Connected | 1 | 2*200 | 1*1 |
| | Layer | | | |
| 13 | Regression | — | — | — |
| | Layer | | | |

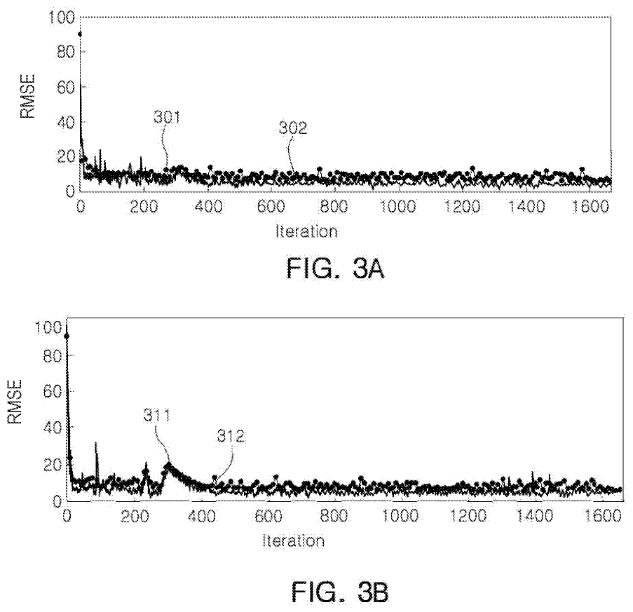

FIGS. 3A and 3B illustrate an error (RSME) between a PG-1T, predicted blood pressure and an actually measured blood pressure value according to an embodiment. FIG. 3A indicates systolic blood pressure (SBP), FIG. 3B indicates diastolic blood pressure (DBP), the X axis may be the number of iteration, the Y axis may be root mean square error (RMVSE), reference numeral 301 may be an actual value of the systolic blood pressure (SBP), reference numeral 302 may be a predicted value of systolic blood pressure (SBP), reference numeral 311 may be an actual value of diastolic blood pressure (DBP), and reference numeral 312 may be a predicted value of diastolic blood pressure (DBP).

As illustrated in FIGS. 3A and 3B, the systolic blood pressure 302 predicted in an embodiment, the actual value 301 of the systolic blood pressure, and RMSE of the diastolic blood pressure 312 and the actual value of the diastolic blood pressure 301 were relatively low, approximately 6.5, indicating that the actual value and the predicted value were almost identical.

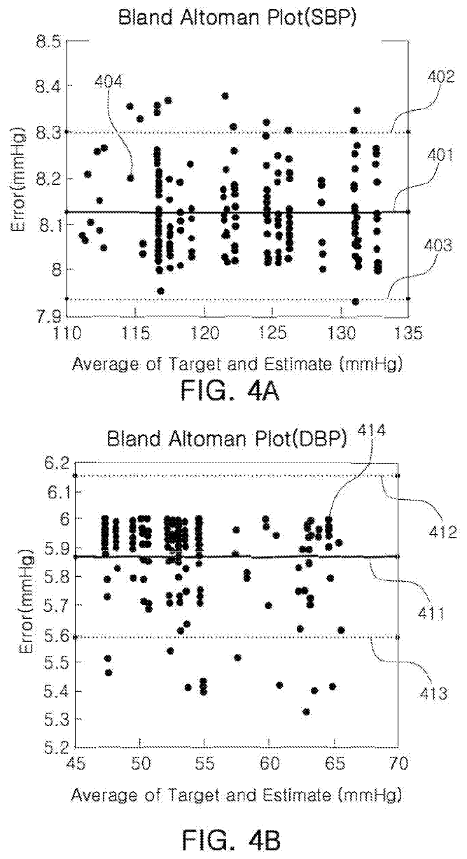

FIGS. 4A and 4B illustrate performance of the apparatus for predicting blood pressure non-compressively using Bland-Altman plot according to an embodiment. FIG. 4A illustrates systolic blood pressure (SBP) and FIG. 4B illustrates diastolic blood pressure (DBP).

The Bland-Altman plot may be a chart illustrating the difference between observation results (predicted values and actual values), and the predicted value may be the predicted systolic blood pressure (SBP) or the diastolic blood pressure (DBP) in an embodiment, and the actual value may be the actual systolic blood pressure (SBP) or the actual diastolic blood pressure (DBP).

The above-described Bland-Altman plot may be obtained in the manner as above.

First, a difference between the actual value and the predicted value may be obtained for each of the systolic blood pressure (SBP) and the diastolic blood pressure (DBP). Thereafter, the mean absolute error (MAE) of the difference values may be obtained according to Equation 3 below.

MAE=average((AV−TV)/ND)) [Equation 3]

In equation 3, the MAE is mean absolute error, AV is actual value, the TV is target value, the ND is number of pieces of data.

Thereafter, the lower limit and the upper limit of each of the systolic blood pressure (SBP) and the diastolic blood pressure (DBP) may be obtained according to Equation 4 below.

LV=MAE−1.96×SD(MAE/ND)

UV=MAE+1.96×SD(MAE/ND) [Equation 4]

In equation 4, the LV is lower limit value, the MAE is mean absolute error, the SD is standard deviation, the ND is number of pieces of data, the UV is upper limit value.

Finally, the values (MAE (401, 411), lower limit values (403, 413), and upper limit values (402, 412) obtained in Equations 1 and 2 may be represented as in FIG. 4. In FIG. 4, solid lines 401 and 411 represent MAE, upper dotted lines 402 and 412 represent upper limit values, lower dotted lines 403 and 413 represent lower limit values, and most of the systolic blood pressure (SBP) 404 predicted according to the present invention were distributed between the upper limit value 402 and the lower limit value 403, or most of the diastolic blood pressure (SBP) 414 predicted according to the present invention were distributed between the upper limit value (412) and the lower limit value 413, it is indicated that the prediction was relatively accurate.

Table 2 below indicates comparison between mean absolute error (MAE) and standard deviation (STD) in the case of using only the existing CNN and in the case of using CNN and LSTM.

TABLE 2

| | | | | SBP | | DBP | |
|---|---|---|---|---|---|---|---|
| | | | | (mmHg) | | (mmHg) | |
| cal-free | Dataset | Model | Input | MAE | STD | MAE | STD |
| PRIOR | MIMICII | CNN | ECG, | 9.30 | 8.85 | 5.95 | 5.52 |
| RESEARCH | | | PPG | | | | |
| PRESENT | BIDMC + | CNN- | ECG, | 8.12 | 8.12 | 5.87 | 7.96 |
| RESEARCH | MIMICII | LSTM | PPG | | | | |

As illustrated in Table 2, in the case of systolic blood pressure (SBP), when only the conventional CNN was used, the MAE was 9.3 and the STD was 8.85, whereas when CNN and LSTM were combined according to an embodiment, the MAE was 8.12. That is, it is indicated that, as the MAE was lower by 1.18, the systolic blood pressure (SBP) was predicted more accurately, which may be the same for diastolic blood pressure (DBP).

As described above, in an embodiment, by predicting blood pressure using an electrocardiogram signal and a pulse wave, there may be an advantage in that blood pressure may be predicted non-compressively.

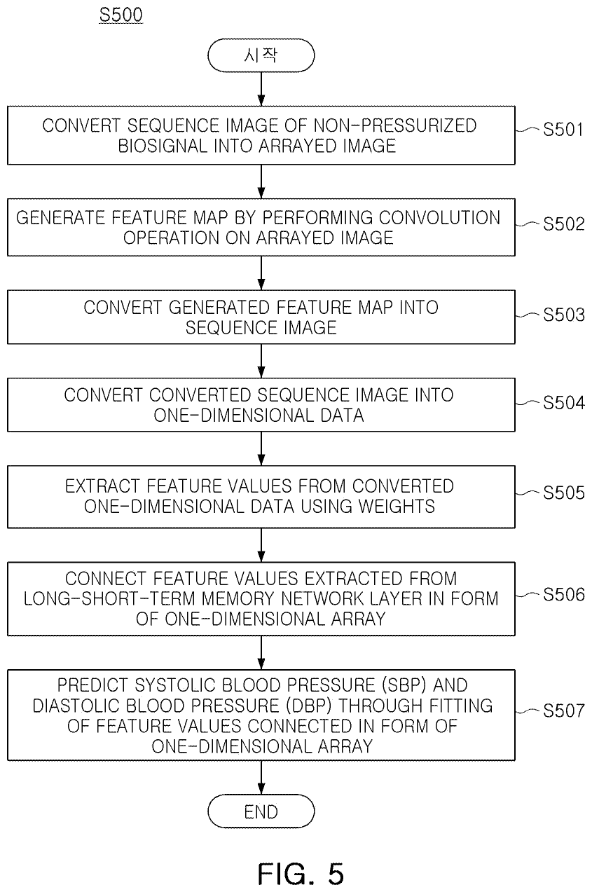

Finally, FIG. 5 is a flowchart illustrating a non-pressurized blood pressure prediction method using a convolutional neural network and a short-term long-term memory network according to an embodiment.

Hereinafter, a method for predicting blood pressure non-compressively using a convolutional neural network and a short-term long-term memory network in an embodiment will be described with reference to FIGS. 1 to 5. For ease of description, descriptions overlapping the descriptions described with reference to FIGS. 1 to 4 will not be provided.

The method for predicting blood pressure non-compressively (S500) in an embodiment may start by converting a sequence image of a non-pressurized biosignal into an arrayed image in a sequence folding layer 120 (S501). The converted array image may be transferred to the CNN layer 130. The aforementioned non-pressurized biosignal may include an electrocardiogram (ECG) signal 111 and a photoplethysmography (PPG) signal.

As described above, the non-pressurized biosignals in the embodiment may be signals 111a and 112a in which the number of pieces of data is increased using random cropping, as illustrated in FIG. 1.

Thereafter, the convolutional neural network layer (CNN) layer 130 may generate a feature map by performing a convolution operation on the arrayed image (S502).

Specifically, the CNN layer 130 may include a first convolution layer 131 generating a first feature map by performing a convolution operation on an arrayed image, a first max pooling layer 133 reducing a dimension of the first feature map by extracting a maximum value of the generated first feature map, a second convolution layer 134 generating a second feature map by performing a convolution operation on the dimensionally reduced first feature map, and a second max pooling layer 136 for reducing a dimension of the second feature map by extracting a maximum value of the generated second feature map.

Also, in an embodiment, a first normalization layer 132 for normalizing the first feature map may be further included between the first convolution layer 131 and the first max pooling layer 133, and as described above, a second normalization layer 135 for normalizing the second feature map may be further included between the second convolution layer 134 and the second max pooling layer 136.

The sequence unfolding layer 140 may convert the generated feature map into a sequence image (S503). The converted sequence image may be transferred to the flatten layer 150.

Thereafter, the flatten layer 150 may convert the converted sequence image into one-dimensional data (S504). The converted one-dimensional data may be transferred to the long-short-term memory network layer 160.

In an embodiment, using one-dimensional data output from the flatten layer as an input value of the long-short-term memory network layer 160, it may not be necessary to convert parameters of the long-short-term memory network layer 160 described later, which may be advantageous.

Thereafter, the long-short-term memory network layer 160 may extract feature values from the one-dimensional data using weights (S505). The extracted feature values may be transferred to the fully connected layer 170.

Thereafter, the fully connected layer 170 may connect the feature values extracted from the long-short-term memory network layer 160 in the form of a one-dimensional array (S506).

Finally, the regression layer 180 may predict systolic blood pressure (SBP) and diastolic blood pressure (DBP) through fitting of feature values connected in the form of a one-dimensional array (S507).

According to an embodiment, by predicting blood pressure using an electrocardiogram signal and a pulse wave, there may be an advantage in that blood pressure may be predicted non-compressively.

The above-described method for predicting blood pressure non-compressively using a convolutional neural network and a long-short-term memory network according to an embodiment described above may be produced as a program to be executed on a computer and may be stored in a computer-readable recording medium. Examples of the computer-readable recording medium may include ROM, RAM, CD-ROM, magnetic tape, floppy disk, optical data storage device, and the like. Also, a computer-readable recording medium may be distributed in a computer system connected through a network, such that the computer-readable code may be stored and executed in a distributed manner. Also, a functional program, a code, and code segments for implementing the method may be easily inferred by programmers in the art to which the present disclosure pertains.

Also, in describing the present disclosure, “ . . . layer” may be implemented by various methods, such as, for example, a processor, program instructions executed by a processor, a software module, a microcode, a computer program product, a logic circuit, an application-specific integrated circuit, firmware, or the like.

While the embodiments have been illustrated and described above, it will be apparent to those skilled in the art that modifications and variations could be made without departing from the scope of the present disclosure as defined by the appended claims.

What is claimed is:

1. An apparatus for predicting blood pressure non-compressively, the apparatus comprising:
a sequence folding layer configured to convert a sequence image of non-pressurized biosignals into an arrayed image;
a CNN layer configured to generate a feature map by performing a convolution operation on an arrayed image;
a sequence unfolding layer configured to convert the generated feature map into a sequence image;
a flatten layer configured to convert the converted sequence image into one-dimensional data;
a long-short-term memory network layer configured to extract feature values from the converted one-dimensional data using weights;
a fully connected layer configured to perform image classification using feature values extracted from the long-short memory network layer; and
a regression layer configured to predict systolic blood pressure (SBP) and diastolic blood pressure (DBP) using the classified image.

2. The apparatus of claim 1, wherein the non-pressurized biosignal is a signal in which the number of pieces of data is increased using random cropping.

3. The apparatus of claim 1, wherein the non-pressurized biosignal includes an electrocardiogram (ECG) signal and a photoplethysmography (PPG) signal.

4. The apparatus of claim 1, wherein the CNN layer includes:
a first convolution layer configured to generate a first feature map by performing a convolution operation on the arrayed image;
a first max pooling layer configured to reduce a dimension of the first feature map by extracting a maximum value of the generated first feature map;
a second convolution layer configured to generate a second feature map by performing a convolution operation on the first feature map of which a dimension is reduced; and
a second max pooling layer configured to reduce a dimension of the second feature map by extracting a maximum value of the generated second feature map.

5. The apparatus of claim 4, further comprising:
a first normalization layer configured to normalize the first feature map between the first convolution layer and the first max pooling layer; and
a second normalization layer configured to normalize the second feature map between the second convolution layer and the second max pooling layer.

6. The apparatus of claim 1, wherein determination of performance of the apparatus for predicting blood pressure non-compressively uses a Bland-Altman plot.

7. A method for predicting blood pressure non-compressively, the method comprising:
a first step of converting a sequence image of non-pressurized biosignals into an arrayed image in a sequence folding layer;
a second step of generating a feature map by performing a convolution operation on an arrayed image in a CNN layer;
a third step of converting the generated feature map into a sequence image in a sequence unfolding layer;
a fourth step of converting the converted sequence image into one-dimensional data in a flatten layer;
a fifth step of extracting feature values from the converted one-dimensional data using weights in a long-short-term memory network layer;
a sixth step of performing image classification using feature values extracted from the long-short-term memory network layer in a fully connected layer; and
a seventh step of predicting systolic blood pressure (SBP) and diastolic blood pressure (DBP) for the classified images in a regression layer.

8. A computer readable storage medium, in which a program for executing the method in claim 7 on a computer is written.
